(19) United States
(12) Patent Application Publication
(10) Pub. No.: US 2010/0331974 A1
(43) Pub. Date: Dec. 30, 2010

(54) Intraocular Kinetic Power Generator

(76) Inventor: Dale Thomas Schaper, JR., Lakeside, TX (US)

Correspondence Address: ALCON, IP LEGAL, TB4-8, 6201 SOUTH FREEWAY, FORT WORTH, TX, 76134, US

(21) Appl. No.: 12/492,255

(22) Filed: Jun. 26, 2009

(51) Int. Cl. A61F 2/14; F03G 7/08; F03G 5/06

(52) U.S. Cl. 623/4.1; 290/1 A

(57) ABSTRACT

An implantable kinetic power generator has an oscillating weight that is physically coupled to a first gear. A second gear with a rotor at its center of rotation is coupled to the first gear. A generating coil is located in close proximity to the rotor. An energy storage unit is electrically coupled to the coil. A shell suitable for implantation in the eye encloses these components. When the rotor rotates, energy generated by the coil is stored in the energy storage unit.

### BACKGROUND OF THE INVENTION

The present invention relates to a power generator for intraocular use and more specifically to a kinetic power generator specifically configured to provide power to an implantable intraocular device.

Anatomically, the eye is divided into two distinct parts—the anterior segment and the posterior segment. The anterior segment includes the lens and extends from the outermost layer of the cornea (the corneal endothelium) to the posterior of the lens capsule. The posterior segment includes the portion of the eye behind the lens capsule. The posterior segment extends from the anterior hyaloid face to the retina, with which the posterior hyaloid face of the vitreous body is in direct contact. The posterior segment is much larger than the anterior segment.

The posterior segment includes the vitreous body—a clear, colorless, gel-like substance. It makes up approximately two-thirds of the eye's volume, giving it form and shape before birth. It is composed of 1% collagen and sodium hyaluronate and 99% water. The anterior boundary of the vitreous body is the anterior hyaloid face, which touches the posterior capsule of the lens, while the posterior hyaloid face forms its posterior boundary, and is in contact with the retina. The vitreous body is not free-flowing like the aqueous humor and has normal anatomic attachment sites. One of these sites is the vitreous base, which is a 3-4 mm wide band that overlies the ora serrata. The optic nerve head, macula lutea, and vascular arcade are also sites of attachment. The vitreous body's major functions are to hold the retina in place, maintain the integrity and shape of the globe, absorb shock due to movement, and to give support for the lens posteriorly. In contrast to aqueous humor, the vitreous body is not continuously replaced. The vitreous body becomes more fluid with age in a process known as syneresis. Syneresis results in shrinkage of the vitreous body, which can exert pressure or traction on its normal attachment sites. If enough traction is applied, the vitreous body may pull itself from its retinal attachment and create a retinal tear or hole.

Various surgical procedures, called vitreo-retinal procedures, are commonly performed in the posterior segment of the eye. Vitreo-retinal procedures are appropriate to treat many serious conditions of the posterior segment. Vitreo-retinal procedures treat conditions such as age-related macular degeneration (AMD), diabetic retinopathy and diabetic vitreous hemorrhage, macular hole, retinal detachment, epiretinal membrane, CMV retinitis, and many other ophthalmic conditions.

A surgeon performs vitreo-retinal procedures with a microscope and special lenses designed to provide a clear image of the posterior segment. Several tiny incisions just a millimeter or so in length are made on the sclera at the pars plana. The surgeon inserts microsurgical instruments through the incisions such as a fiber optic light source to illuminate inside the eye, an infusion line to maintain the eye's shape during surgery, and instruments to cut and remove the vitreous body.

Another common surgical procedure, cataract removal and lens replacement, is performed on the anterior segment of the eye. The eye's natural lens is composed of an outer lens capsule enclosing a lens cortex. Since the human eye functions to provide vision by transmitting light through a clear outer portion called the cornea, and focusing the image by way of a clear crystalline lens onto a retina, the quality of the focused image depends on many factors including the transparency of the lens. When age or disease causes the lens to become less transparent, vision deteriorates because of the diminished light which can be transmitted to the retina. This deficiency in the lens of the eye is medically known as a cataract. An accepted treatment for this condition is cataract surgery which involves the removal and replacement of the lens cortex by an artificial intraocular lens (IOL).

In the United States, the majority of cataractous lenses are removed by a surgical technique called phacoemulsification. During this procedure, an incision of a few millimeters in size is made in the cornea or sclera. By way of the incision, a thin phacoemulsification cutting tip is inserted into the diseased lens and vibrated ultrasonically. The vibrating cutting tip liquefies or emulsifies the lens cortex material so that it may be aspirated out of the eye. The diseased lens material, once removed, is replaced by an IOL.

The IOL is injected into the eye through the same small incision used to remove the diseased lens cellular material. The IOL is placed in an IOL injector in a folded state to avoid enlarging the incision. The tip of the IOL injector is inserted into the incision, and the lens is delivered into the lens capsular bag.

During these procedures and others, an implant is sometimes placed in the eye. For example, vitreo-retinal surgery may result in the placement of an electronic retina device. While currently not commercially available, a great deal of work has been devoted to developing an electronic replacement for a damaged retina. Since the retina is essential for sight, damage to the retina often results in the loss of sight. An electronic replacement for a retina has been conceptually developed and tested at the University of Southern California (Argus II Retinal Prosthesis System).

Work is also being done on an accommodative IOL. An accommodative IOL simulates the movement of the natural lens when a person focuses his eyes. As such, a single natural lens can be adjusted to focus on objects very close to the face or on objects at a great distance. In some accommodative IOL concepts, a power source is required to adjust the IOL to simulate the ability of the eye. Other devices, like implantable drug delivery devices, may also require power.

These devices, and others like them, need a reliable source of power in order to function. Current battery technology is usually not adequate in that batteries, once implanted in the eye, may have to be removed and replaced. Further, the amount of power that can be delivered on a single battery charge typically does not power an implantable device for an extended period of time. What is needed is a better source of power for such implanted ocular devices.

### SUMMARY OF THE INVENTION

In one embodiment consistent with the principles of the present invention, the present invention is an implantable kinetic power generator. The generator has an oscillating weight with a curved profile such that a center of rotation of the oscillating weight is not coplanar with a periphery of the oscillating weight. A first gear is physically coupled to the oscillating weight such that a center of the gear is located at the center of rotation of the oscillating weight. A second gear with a rotor at its center is coupled to the first gear. A generating coil is in close proximity to the rotor. An energy storage unit is electrically coupled to the coil. A shell encloses the oscillating weight, the first gear, the second gear, the rotor, the generating coil, and the energy storage unit. The shell has a curved profile such that a center of the shell is not coplanar with a periphery of the shell thereby making the shell suitable for implantation into the eye.

In another embodiment consistent with the principles of the present invention, the present invention is an implantable kinetic power generator. The generator has an oscillating weight that is physically coupled to a first gear. A second gear with a rotor at its center of rotation is coupled to the first gear. A generating coil is located in close proximity to the rotor. An energy storage unit is electrically coupled to the coil. A shell suitable for implantation in the eye encloses these components. When the rotor rotates, energy generated by the coil is stored in the energy storage unit.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a perspective view of the components of an intraocular kinetic power generator according to the principles of the present invention.

FIG. 2 is a cross section view of the components of an intraocular kinetic power generator according to the principles of the present invention.

FIG. 3 is a perspective view of a kinetic power generator as implanted in the eye according to the principles of the present invention.

### DETAILED DESCRIPTION OF THE PREFERRED EMBODIMENTS

Reference is now made in detail to the exemplary embodiments of the invention, examples of which are illustrated in the accompanying drawings. Wherever possible, the same reference numbers are used throughout the drawings to refer to the same or like parts.

The inventor has found that a kinetic power generator suitable for intraocular use can be based on the kinetic power generator used in wrist watches. Kinetic wrist watches use the motion of the human body (in this case, the swinging arm) to generate the power required for use by the watch. Seiko first introduced kinetically powered wrist watches in the 1980's. Since that time, a number of different watch companies have marketed wrist watches that run on kinetically generated power. The benefit of these kinetically powered wrist watches is that they do not have batteries that need to be replaced. This feature makes a kinetic power generator particularly useful for an intraocular implant.

While based on known kinetic power generators, the present invention utilizes some specific modifications that render the kinetic power generator particularly suitable for use in the eye. These modifications are structural in nature and are more fully described with reference to the Figures.

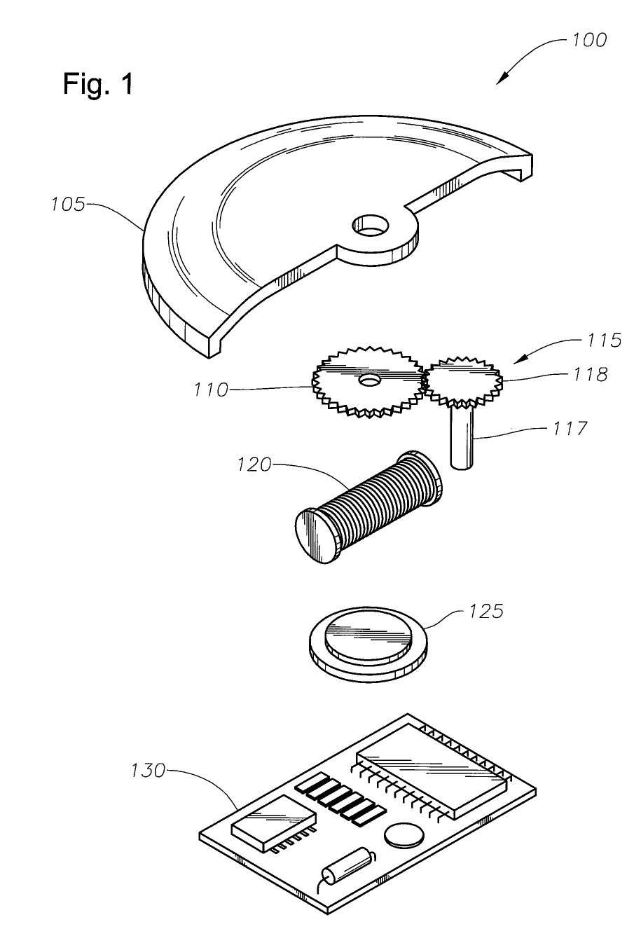

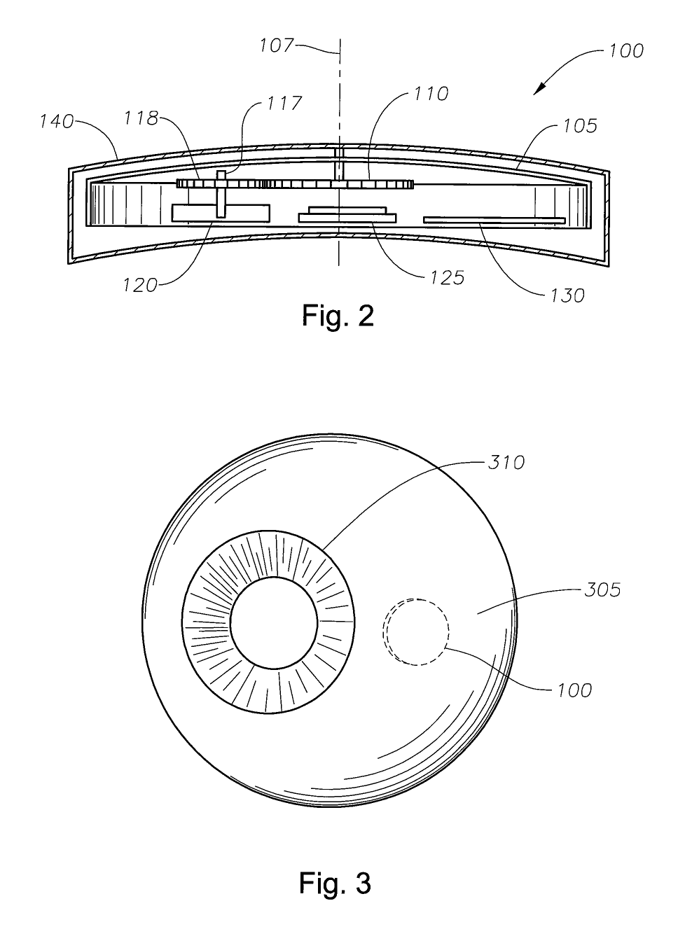

FIG. 1 is a perspective view of the components of an intraocular kinetic power generator 100 according to the principles of the present invention. FIG. 2 is a cross section view of the components of an intraocular kinetic power generator 100 according to the principles of the present invention. In FIGS. 1 and 2, oscillating weight 105 is coupled to gear 110. Gear 110 interfaces with geared rotor 115. Geared rotor 115 is magnetically coupled to generating coil 120. Generating coil 120 is electrically coupled to energy storage unit 125. A control circuit 130 is electrically coupled to energy storage unit 125.

Oscillating weight 105 is generally in the shape of a semi circle and has a slightly curved profile. The slightly curved profile accommodates the curvature of the eye and allows oscillating weight to be contained in a shell 140. In this slightly curved profile, the center of rotation 107 is not coplanar with the periphery of oscillating weight 105. Oscillating weight 105 is made of a material with a relatively substantial mass such as stainless steel. In addition, mass is concentrated along the periphery of oscillating weight 105 furthest from the center of rotation 107. The concentration of mass in this manner allows for oscillating weight 105 to efficiently enable the production of kinetic energy.

Oscillating weight 105 is typically a few millimeters in diameter (as measured across a diameter of the semi-circle). Despite its small size, the natural movement of the eye causes oscillating weight 105 to rotate. Natural movement of the eye in the azimuth and elevation directions provides the necessary energy to cause oscillating weight 105 to rotate. The eye naturally moves at a relatively high rotational velocity and with relatively high rotational acceleration. Further, the radius of the moment of inertia is also small. Therefore, oscillating weight 105 can be small as well and still produce sufficient kinetic energy to power an intraocular device.

Oscillating weight 105 is physically connected to gear 110 such that rotation of oscillating weight 105 results in rotation of gear 110. Gear 110 is configured to amplify the rotational movement of oscillating weight 105 by up to a factor of 100 or more. The center of gear 110 and the center of rotation 107 of oscillating weight 105 are at the same point. In one embodiment of the present invention, gear 110, like oscillating weight 105, has a slightly curved profile so as to fit in shell 140 and accommodate the curvature of the eye. In this manner, the center of gear 110 is not generally coplanar with the periphery of gear 110. In other embodiments of the present invention, gear 110 is generally flat.

Gear 110 is coupled to geared rotor 115. Again, the gear ratio is such that movement of gear 110 is amplified. As such, geared rotor 115 spins much faster than gear 110. Geared rotor 115 has a central rotor 117 located at the center of a gear 118. The teeth of gear 110 interface with the teeth of gear 118. Since central rotor 117 is located at the center of rotation of gear 118, movement of gear 118 causes a very rapid rotation (on the order of 100,000 rpm) of central rotor 117. Central rotor 117 is made of samarium cobalt so that a magnetic field is generated when it rotates.

Generating coil 120 is located very close to central rotor 117 such that a magnetic field generated by central rotor 117 (as it rotates) generates an electrical current in generating coil 120. Generating coil 120 is an extremely high density coil wound with very thin wire, such as extremely small gauge copper wire. In the presence of a magnetic field, generating coil 120 produces a small current. Generating coil 120 may also have a slightly curved profile so that it fits into shell 140. Further, generating coil is typically wound around a flat core so as to keep its overall height very small (on the order of a fraction of a millimeter). Such a small height is desirable for implantation in the eye.

Generating coil 120 is electrically coupled to energy storage unit 125. In one embodiment of the present invention, energy storage unit 125 is a super capacitor that stores a charge sufficient to provide power to an intraocular device. In another embodiment of the present invention, energy storage unit 125 is a very small rechargeable battery, capacitor, or similar type of device.

Controller 130 controls the operation of the kinetic power generator. Controller 130 is typically an integrated circuit with power, input, and output pins capable of performing logic functions. In various embodiments, controller 130 is a targeted device controller. In such a case, controller 130 performs specific control functions targeted to a specific device or component, such as power control functions for the kinetic power generator or power transfer functions to an intraocular device. In other embodiments, controller 130 is a very small microprocessor. In such a case, controller 130 is programmable so that it can function to control more than one component of the device. In other cases, controller 130 is not a programmable microprocessor, but instead is a special purpose controller configured to control different components that perform different functions.

Shell 140 is sized and shaped to fit on the eye. In this manner, shell 140 is generally circular in shape and has a curved profile. As such a center of shell 140 is not coplanar with the periphery of shell 140. This curvature (like the curvature of oscillating weight 105) is designed to conform to the curvature of the eye. Shell 140 is typically made of stainless steel or other material suitable for implantation into the eye.

In addition, the location of the various components in shell 140 is such that the overall height of shell 140 is minimized. As noted, generating coil 120 is wound on a generally flat core so as to minimize its height. Further, rotor 117, gear 110, generating coil 120, and energy storage unit 125 are all located on approximately the same plane. The parallel location of these components allows the height of shell 140 to be on the order of one millimeter.

In operation, the intraocular kinetic power generator 100 of the present invention transforms the natural movement of the eye into electrical power to run an intraocular device. The human eye moves a significant portion of the time. When a person is awake, his eyes make both small and large rotational movements. When asleep, significant eye movement also occurs (for example, in REM sleep). This movement of the eye during both night and day provides ample kinetic energy to run the kinetic power generator of the present invention.

As the eye moves (or rotates), the kinetic power generator also moves. This movement of the eye causes oscillating weight 105 to rotate. Since gear 110 is rigidly coupled to oscillating weight 105, gear 110 also rotates. As noted, the teeth of gear 110 are coupled to the teeth of gear 118. Therefore, rotation of gear 110 causes rotation of gear 118 (and the central rotor 117). The gear ratio is such that central rotor 117 rotates very rapidly thereby producing a magnetic field. Since central rotor 117 is located very close to generating coil 120, the magnetic field produced by central rotor 117 couples with generating coil 120 thereby producing a small current in generating coil 120 (and a small voltage across generating coil 120). Energy produced by generating coil 120 is stored in energy storage unit 125. This stored energy can be used to run an intraocular device.

Once implanted in the eye, an intraocular kinetic power generator 100 can theoretically last the lifetime of the patient. Because of this, only a single operation is required. Since any operation, and particularly an operation on the eye, presents the risk of complications including infections, it is desirable to minimize the number of operations a patient has. By implanting a device with a kinetic power generator instead of a traditional battery, a single operation can implant the device. The natural movement of the eye can then be harnessed to generate sufficient power to run the device.

FIG. 3 is a perspective view of an intraocular kinetic power generator 100 as implanted in the eye according to the principles of the present invention. The kinetic power generator of the present invention is typically implanted in or under the sclera 305 or under the conjunctiva posterior to the limbus 310. If implanted in or under the sclera 305, an incision is made in the sclera 305 (like a scleral flap). The incision is typically a few millimeters in length. A pocket is formed in or under the sclera 305 to receive the kinetic power generator. Likewise, a similar procedure is performed for implantation under the conjunctiva.

From the above, it may be appreciated that the present invention provides a reliable and steady source of power for implantable ocular devices. The present invention provides a kinetic power generated specifically configured for the eye. The present invention is illustrated herein by example, and various modifications may be made by a person of ordinary skill in the art.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An implantable kinetic power generator comprising:
an oscillating weight with a curved profile such that a center of rotation of the oscillating weight is not coplanar with a periphery of the oscillating weight;
a first gear physically coupled to the oscillating weight, such that a center of the gear is located at the center of rotation of the oscillating weight;
a second gear with a rotor at a center of rotation of the second gear, the second gear coupled to the first gear;
a generating coil in close proximity to the rotor;
an energy storage unit electrically coupled to the coil; and
a shell enclosing the oscillating weight, the first gear, the second gear, the rotor, the generating coil, and the energy storage unit;
wherein the shell has a curved profile such that a center of the shell is not coplanar with a periphery of the shell, and further wherein the shell is suitable for implantation into the eye.

2. The kinetic power generator of claim 1 wherein the first gear has a curved profile such that a center of the first gear is not coplanar with a periphery of the first gear.

3. The kinetic power generator of claim 1 wherein the energy storage unit is a super capacitor.

4. The kinetic power generator of claim 1 further comprising:
a controller for controlling power functions of the kinetic power generator.

5. The kinetic power generator of claim 1 wherein the shell is suitable for implantation under the sclera.

6. The kinetic power generator of claim 1 wherein the generating coil is wound around a flat core so as to minimize its height.

7. The kinetic power generator of claim 1 wherein the generating coil is located adjacent to and generally coplanar with the rotor.

8. The kinetic power generator of claim 1 wherein the generating coil is located generally coplanar with the energy storage unit.

9. An implantable kinetic power generator comprising:
an oscillating weight;
a first gear physically coupled to the oscillating weight;
a second gear with a rotor at a center of rotation of the second gear, the second gear coupled to the first gear;
a generating coil in close proximity to the rotor;
an energy storage unit electrically coupled to the coil; and
a shell enclosing the oscillating weight, the first gear, the second gear, the rotor, the coil, and the energy storage unit;
wherein the shell is suitable for implantation into the eye.

10. The kinetic power generator of claim 9 wherein the energy storage unit is a super capacitor.

11. The kinetic power generator of claim 9 further comprising:
a controller for controlling power functions of the kinetic power generator.

12. The kinetic power generator of claim 9 wherein the shell is suitable for implantation under the sclera.

13. The kinetic power generator of claim 9 wherein the generating coil is wound around a flat core so as to minimize its height.

14. The kinetic power generator of claim 9 wherein the generating coil is located adjacent to and generally coplanar with the rotor.

15. The kinetic power generator of claim 9 wherein the generating coil is located generally coplanar with the energy storage unit.
